(19) United States
(12) Patent Application Publication
(10) Pub. No.: US 2004/0225174 A1
(43) Pub. Date: Nov. 11, 2004

(54) Method for computed tomography-ultrasound interactive prostate brachytherapy

(76) Inventors: Donald B. Fuller, Rancho Santa Fe, CA (US); Haoran Jin, San Diego, CA (US)

Correspondence Address: GORDON & REES LLP, 101 WEST BROADWAY, SUITE 1600, SAN DIEGO, CA, 92101, US

(21) Appl. No.: 10/430,547

(22) Filed: May 6, 2003

(51) Int. Cl. A61N 5/00; G06F 17/60; G06F 19/00; G01N 33/48; G01N 33/50

(52) U.S. Cl. 600/1; 702/19; 705/3

(57) ABSTRACT

The invention is a method for the treatment of cancer comprising interactive imaging techniques to optimize the placement of radioactive seeds in a portion of the body containing cancer. The method comprises the computer assisted development of a seed placement plan based on images obtained on a pre-operative day. Seeds are placed into the region of interest in the body, typically the prostate, using an ultrasound guided method. The seeds are imaged using computed tomography (CT) to assess the dose distribution. A second plan, or miniplan, is developed based on information obtained in the CT scan. Additional ultrasound-guided seeds are placed at the same procedure based on the miniplan to optimize the final dose coverage.

### BACKGROUND OF THE INVENTION

Prostate cancer is a serious health concern, with approximately 180,000 new cases diagnosed each year in the United States and 396,000 reported worldwide. (D. C. Beyer, Cancer Control, 8:163-170, 2001) With the widespread use of prostate specific antigen (PSA) screening over the past decade, patients are being seen with earlier stage disease. Cure rates are rapidly improving, with 5-year survival rising from 67% in 1976 to 93% in 1994.

At the same time, the number of therapeutic options available has increased. Permanent brachytherapy, commonly called “seed implantation” has become an acceptable standard means of therapy and is now available to radiation oncologists and urologists worldwide. Brachytherapy is the placement of radioactive sources in close proximity to any tumor. It takes advantage of the simplest physical property of radiation. High doses of radiation are present in the vicinity of the radioactive material, but the dose decreases with the square of the distance from the source. Prostate brachytherapy has been performed over the years with a variety of approaches with methods improving as new technologies become available. Techniques have evolved from intreaurethral insertion of a temporary source, to permanent interstitial implantation using a retropubic approach, and eventually to the current ultrasound guided transperineal technique.

Initial implantation techniques were limited by the ability to place seeds in an appropriate manner resulting in poor source and dose distribution and high recurrence rates. The technique proved to be difficult to reproduce widely and many implants were judged to be inadequate. In 1983, Holm et al (J. Urol. 3:283-6) described the use of transrectal ultrasonography to guide transperineal insertion of needles into the prostate to permanently deposit ¹²⁵iodine (I) sources into the gland. This method has been modified and optimized by a number of individuals as demonstrated both by the popularity of the therapy and a number of publications in medical journals.

A variety of techniques have been developed and are in current practice; however, the basic steps of the method are consistent, regardless of the specifics of the technique. Implantation is almost always performed as minor outpatient surgery under general or spinal anesthesia. An implant typically requires approximately one hour, and patients can return home after a brief recovery. In an effort to achieve optimal geometry of the implanted sources, templates are almost universally used, in contrast to the freehand approach commonly used with retropubic implantation. With the patient in the lithotomy position, templates are held rigidly in place over the perineum and act as guides for needle placement. This allows for control over the entire prostate target volume and specification of source placement at any point within the gland. If the prostate is imagined as a three dimensional ellipsoid within the pelvis, then any point within the prostate can be given a unique set of coordinates using the grid on the template to define the X and Y coordinates and using the distance from the plane at the base of the prostate to define the Z coordinate. For every 5-mm increment or “step” from the “zero plane” at the base of the prostate to the apex, transrectal ultrasonography can be used to map the area of the gland onto the grid. This creates a series of 2-dimensional images that can be totaled to create a 3-dimensional target volume. For a number of reasons, the treated area is slightly larger than the actual organ boundaries. While no absolute margin is applied, the general consensus has developed that 3-5 mm is generally adequate and can be achieved.

Treatment planning software programs, such as Variseed 7.0 as well as those marketed by ADAC (Philips), Prowess and Burdette Medical Systems have been developed to plan a target volume and to develop a pattern for radioactive source placement that will deliver the desired dose. The position of each source is defined based on the grid coordinate system that follows from the chosen template, and its depth or distance from the template. Initially, regular spacing between seeds was used; however, with improved planning capabilities, irregular spacing is more commonly used. Planning may take place weeks in advance or may be performed intraoperatively.

It is generally agreed that postoperative dosimetry must be performed to assess the adequacy of the implantation and to determine the actual dose received by the prostate and the normal tissue. Historically, this dose calculation was performed using a pair of orthogonal radiographs. It was possible to identify those implants with significant dose inhomogeneity and recognize those patients at higher risk for treatment failure. However, these films fail to identify the prostate anatomy. With the more recent use of computed tomography (CT) scanning for dose calculation, it is possible to calculate the dose received by the prostate more accurately.

Within the gland, some dose inhomogeneity is always produced. As a result, the dose is generally defined to a volume rather than at one fixed point. A variety of dose measures have been recognized, such as the D90 (dose received by 90% of the prostate volume) of V100 (percent volume of the prostate receiving the prescription dose). These dose parameters are of increasing importance as a quality indicator for the brachytherapist and a prognostic indicator for the patient. While controversy continues regarding many technical details, such as when to perform the CT scan and how to define the prostate contour, there is evidence that these parameters predict outcome. (R. G. Stock et al., Int. J. Radiat. Oncol. Biol. Phys., 48:899-906, 2000; L. Potters et al., Int. J. Radiat. Oncol. Biol. Phys., 50:605-14, 2001)

Though it is relatively simple to to produce an optimal plan for prostate brachytherapy, the actual final result of the procedure may be considerably degraded due to seed migration, needle deviation, bone interference, prostate swelling, bleeding and difficulty imaging. One or more of these factors may unpredictably contribute to produce an inadequate brachytherapy radiation dose distribution in any patient causing the need for an additional treatment, which is traumatic and costly, or decreasing their probability of a cure, which is also problematic.

The lack of an effective method to analyze and correct dosimetry shortcomings during the procedure is one of the largest technical problems of prostrate brachytherapy. To address this need, ultrasound-based and magnetic resonance imaging (MRI)-based real-time seed adjustments and dosimetery methods have been described. (A. V. D'Amico et al., J. Endourol. 14:367-70, 2000, D. C. Beyer et al., Int. J. Radiat. Oncol. Biol. Phys. 48:1583-9, 2000) Unfortunately, though both of these imaging modalities reasonably identify the appropriate urologic anatomy, they both are limited in the ability to accurately identify every implanted source, and also potentiality visualize densities that are not sources, mistakenly identifying them as sources. This fundamental source identification specificity and sensitivity issue limits the accuracy and utility of these methods. Additionally, the MRI-based method requires the use of an open MRI and suffers from being expensive, time consuming and scarcely available. Intraoperative fluoroscopy has also been used to augment the basic ultrasound guidance method, but imaging using fluoroscopy requires that the needles be left in for imaging so that the two images may be aligned as the prostate contours cannot be detected by fluoroscopy and the sources cannot be accurately detected by ultrasound (Gong et al., Int J. Radiat Oncol Biol Phys. 54:1322-30, 2002). The clinical utility of method remains unproven, particularly when a large number of sources have been implanted.

### SUMMARY OF THE INVENTION

The invention is a method for real-time dosimetery analysis of brachytherapy using computed tomography (CT) imaging. The invention is the first use of CT-imaging in “real-time” during the surgical process to allow for correction of deficiencies during the procedure. CT-imaging is readily and rapidly available, gives a reasonable definition of relevant urologic anatomy and is a far more accurate method of source identification than ultrasound or MRI. CT allows imaging of the prostate which is not possible in fluoroscopy. The method of the invention comprises fusing the CT-generated isodose lines with an ultrasound guidance template, and based on this specific feedback, directly planning and accomplishing an immediate CT-ultrasound interactive brachytherapy improvement during the same procedure, resulting in an improved final brachytherapy product.

Briefly, a brachytherapy preplan is developed most frequently based on pre-operative ultrasound images. Seed placement is performed by any acceptable method, typically using a template guide to define coordinates within the prostate. Following completion of the placement of all sources specified by the preplan, the patient has an immediate stepping ultrasound volume study and the images are transferred to a workstation where ultrasound based contouring is accomplished. Simultaneously, the patient travels through a CT-scanner as rapidly as possible and is returned to the operating suite and re-prepped by the nursing staff. The CT-images are sent to the same workstation as the ultrasound scans and CT-based contouring is accomplished. CT automated source identification and dosimetry analysis follow. The images are fused by overlaying the ultrasound-based and CT-based contours and aligning them as accurately as possible in three planes. Any dosage deficiencies are noted. Simulated seeds are added to the overlaid images relative to the image fused ultrasound grid, and analysis is performed to determine the number and location of additional seeds to compensate for any deficiencies in the initial placements or to increase dosage in desired regions (e.g. at major lesions). If necessary, a mini-plan is developed for implantation of additional seeds. Ideally, the imaging, initial dosimetry calculation, image fusion, development of a mini-plan, evaluation and seed placement should add approximately 45-60 minutes to the overall procedure time.

### BRIEF DESCRIPTION OF THE DRAWINGS

FIG. 1 is a schematic of overlaid optical sections of a prostate showing an ultrasound contour (dashed line), a CT contour (solid line) and implanted seeds (filled-in circles) on a grid;

FIG. 2 is the overlaid optical sections of FIG. 1 with calculated dose lines (alternating dot-dash line) relative to the prostate contours;

FIG. 3 is the overlaid optical section of FIG. 2 with the contour and dose lines with the dosing deficiencies indicated;

FIG. 4 is the overlaid optical sections of FIG. 3 overlaid on an ultrasound template guidance grid to allow definitive identification of locations of underdosage;

FIG. 5 is a schematic of an overlaid optical section obtained close to the section used for FIGS. 1 though 4 showing the altered dose lines from the insertion of additional seeds; and

FIG. 6 is a graph showing the V100 after the first and second rounds of seed implantation and the percent improvement from first to second round of seeding.

The present invention will be better understood from the following detailed description of an exemplary embodiment of the invention, taken in conjunction with the accompanying drawings.

### DETAILED DESCRIPTION AND PREFERRED EMBODIMENT

Computed tomography or CT is an imaging method in which the region of interest is imaged by taking serial, parallel sections of the region at regular intervals followed by digital reassembly of the sections to provide a three dimensional image. CT can be used to image soft tissue, bone, vasculature and implanted seeds. CT scanners are available from a number of vendors known to those skilled in the art including General Electric and Seimans.

A D value is a measure of the dose of radioactivity received by a specific percentage of the prostate. For example, D90 means that 90% of the prostate has received a given dose. This number is obtained by a computer calculation of the radiation isodose line that covers exactly 90% of the contoured prostate volume.

Dosimetry is analysis of a region of the body to which radioactivity was delivered and to determine the quantity and distribution of the radioactivity.

A Gray (145Gy) is a dose of radiation equal to 100 rads.

A grid is a reference system of points placed at 5 mm intervals. The guidance template is made of plastic and has 169 holes through which brachytherapy needles may be guided to grid-referenced target points within the prostate.

An isotope in the context of this invention, is a radioactive molecule. Those used for the method of the invention include, but are not limited to ¹⁰³Pd (palladium), ¹²⁵I (iodine) and ¹⁹⁸Au (gold).

A miniplan is a seed placement plan for the second round of seed placements. It is developed based on information obtained after imaging and interval dosimetry analysis which is performed after the first round of seed placements. A minority, typically no more than 20%, of the radioactivity is placed based on this plan.

An optical section is a thin slice through the body taken using an imaging device. The slices are assembled in a computer to render a three dimensional image. In the method of the invention, optical sections are typically obtained every 3-5 mm.

A preplan is a seed placement plan developed based on imaging of the prostate before any seed implantation. A majority of the radioactivity, typically at least 80%, is placed based on this plan.

The planning target volume or PTV is the volume of the prostate plus appropriate margin as determined using 3 dimensional imaging methods. There is some variance in the volume determined using various imaging methods.

Real-time imaging takes place during the procedure as opposed to before the procedure for planning purposes or after the procedure to determine the quality of the result.

A seed or source is a commercially available radioactive pellet containing the isotope of choice. They are approximately 4.5 mm long and 1 mm wide and can be delivered to a site of interest in the body using a needle.

The simulated dose distribution is the calculated dose of radioactivity to the region of interest based at least partially on seeds that have not yet been placed in the body.

A simulated seed is a point source of radioactivity that is placed into an image to allow for a theoretical calculation of dose distribution. Typically simulated seeds are placed into images obtained after the first round of seed placements which contain a number of actual radioactive sources to ensure that the addition of the supplemental seeds in the second round will result in the desired outcome.

Ultrasound imaging is a method to visualize both soft tissue and bone with a moderately high resolution. In the case of prostate brachytherapy, the prostate, urethra and seminal vesicles are imaged. Ultrasound is typically used for guidance during the seed implantation procedure as it can be used to image needle placements in relationship to the prostate. Imaging ultrasounds are available from a number of vendors known to those skilled in the art including Siemans, General Electric, B&K and Acuson.

A V value is the volume of the prostate that receives a specified dose. For example, a V100 of 99% means that 99% of the entire prostate received 100% of the prescribed dose.

### DETAILED DESCRIPTION

The invention is a method for real time CT-ultrasound interactive brachytherapy for the treatment of cancer in any tissue sufficiently close to the surface of the body which is sufficiently solid to allow for placement of seeds through the skin without making an incision. The method is preferably designed for the treatment of prostate cancer.

A patient diagnosed with prostate cancer is selected for prostate brachytherapy. On a pre-operative day, the patient is subjected to a series of tests including a stepping ultrasound volume study to determine the contour of the prostate and develop a patient specific seed placement preplan.

On the operative day, a patient specific modified peripheral pre-planned, pre-loaded prostate brachytherapy technique is accomplished using any of a number of methods, software programs and imaging apparatuses well known to those skilled in the art. The selection of software and a CT scanner is a routine matter of choice frequently made by those skilled in the art. The selection is made based on a number of criteria including, but not limited to, the availability of equipment and the state of the art. Preferably, the technique is accomplished using Variseed® software (Version 7.0 or higher) and Siemens Sonoline Prima® multiplane, megahertz ultrasound equipment, based on their conventional stepping ultrasound-based volume plus a 3-5 mm margin. This region is defined as the planning target volume (PTV). The procedure is carried out using standard ultrasound guided technique per pre-plan specification in the operating suite.

The method of the invention deviates from standard brachytherapy techniques in that the patient is typically under conscious sedation with generous local anesthetic rather than general anaesthesia. This facilitates the procedure. However, the method may also be carried out using spinal anesthesia and the exact method of sedating the patient is not a limitation of the method of the invention.

Following completion of the placement of all sources specified in the preplan, the patient has an immediate stepping ultrasound volume study, using the ultrasound device used in the seed placement procedure, and each of the images is transferred to the brachytherapy workstation where ultrasound based contouring is then accomplished on the computer monitor. This reveals any changes in the prostate contour and volume that may have taken place due to bleeding or swelling. While the contouring is being performed by the radiation oncologist or other qualified individual, the patient is simultaneously transferred to the CT suite. A CT-scan is performed in any appropriate scanner such as a GE 9800 quick CT scanner. The CT scan is performed at scan increments that match the ultrasound scan increments and the images are exported immediately to the brachytherapy workstation. The images are assembled to allow for source identification, contour analysis and dosimetry analysis by the radiation oncologist or other individual with the appropriate skill in the art. As soon as the scan is completed, the patient is transferred back to the operating suite and re-prepped by nursing staff.

At the workstation, the ultrasound and CT-images are analyzed to develop a miniplan. More specifically, after the ultrasound-based prostate contouring, source identification, CT-based prostate contouring and CT urethral contouring are completed; the CT-based isodose plan is generated and critically analyzed for cold spots relative to the CT contoured prostate volume. The ultrasound and CT prostate volumes are then co-registered, typically using the same number of incremental optical sections using appropriate brachytherapy image fusion software. The ultrasound and CT contour sets are displayed, typically in different colors on the computer monitor, and aligned as closely as possible in all directions, cephalad, cardad, anteriorly, laterally and posterior-laterally, accepting that the posterior-central alignment will be imperfect in the case of prostate treatment, due to the condition of the rectal probe causing some central prostate distortion on the ultrasound images which is not present on corresponding CT-images. Typically, the alignment process is within 2-3 mm, though potentially larger deviations appear possible directly posteriorly, due to ultrasound probe distortion of the posterior prostate surface.

Upon completion of the ultrasound-CT registration process, the dosimetry lines are displayed directly over the image-fused ultrasound grid template images, to identify potential areas of underdosage relative to the CT and ultrasound prostate contours, as well as reference to the ultrasound template guidance grid. Areas where the prescription isodose line either enters or closely approaches the planning target volume are identified on the appropriate optical sections, relative to the image-fused ultrasound grid. This distance of the most proximal aspect of each of these areas from the base of the prostate is then measured and recorded. The number of seeds per coordinate to correct the problem is also estimated and recorded. This identification and coordinate-by-coordinate correction process is accomplished until every area of concern has been addressed.

Once the above process has been completed, a mini-plan is created using the information obtained from the above image alignment process to correct any deficiencies. Ideally, no more than 15% additional millicuire activity is used in favorable prognosis cases and no more than 20% additional millicurie activity is used in poor prognosis cases. The seeds are typically divided into needles loaded with two to four seeds per needle.

The reasons for greater potential millicurie activity in non-favorable cases are three-fold. First, it is judged even more important to comprehensively eradicate cold spots. Second, sources are more likely to be placed in more remote locations (e.g. extra-prostatic locations in the case of prostate cancer), to ensure wide coverage of the dominant lesion or lesions, increasing the number of sources required. Third, palladium-103 (¹⁰³Pd) sources are often used for less favorable cases and this isotope is more likely to result in cold spots due to its low energy as compared to ¹²⁵I which is used commonly in cases with a favorable prognosis.

At the discretion of the radiation oncologist or other skilled individual, all supplemental sources may be implanted even when the initial dosimetry analysis result does not require all of them for correction. In instances where the full additional millicurie activity does not appear necessary to correct dosimetry effects, the extra sources can be implanted into the far lateral peripheral zone of the prostate, particularly on the side of the dominant lesion or lesions, to bulge the isodose lines a bit further beyond the prostate, taking care to keep additional sources away from the urethra and rectum. In the non-favorable cases, in addition to the standard prostate brachytherapy pattern, stranded sources may be implanted into the proximal seminal vesicles and peri-prostatic tissues around the dominant lesion or lesions.

At the conclusion of the development of the miniplan, the information is inserted into the seed map generated during the real time imaging using an indicator such as a different font. A calculation is made to insure that the additional seeds will result in a final outcome that is satisfactory to the surgeon (e.g. cold spots eliminated, additional seeds added to dominant lesions). If the simulated seed placements do not result in the desired final outcome, the simulated placements are manipulated within the image until a satisfactory miniplan has been developed.

In the surgical suite, the radiation oncologist, anesthesiologist or other skilled individual reassesses the adequacy of the anesthesia before proceeding with the additional source placement. If needed, additional anesthesia is administered, and the procedure is subsequently completed. The supplemental seeding based on the miniplan is substantially more rapid than the initial seeding procedure, typically requiring only 5-15 minutes.

Theoretically, the patient could be subjected to another round of imaging and implantation, but it is unlikely that a substantial increase in therapeutic value of the intervention would be obtained.

The patient is subjected to a post-brachytherapy simulation film in which two x-rays are taken perpendicular to each other, and a CT-based dosimetry analysis, preferably on the same day, before discharge from the office. The real-time CT-based technique requires two CT studies instead of one, and increases the total brachytherapy procedural time by an average of about 45-60 minutes compared with a standard non-real time CT-ultasound interactive dosimetery guided procedure. However, by performing the imaging that would typically be done in a follow-up visit, time is saved overall. Most importantly, it improves the final seed placement which improves patient outcome.

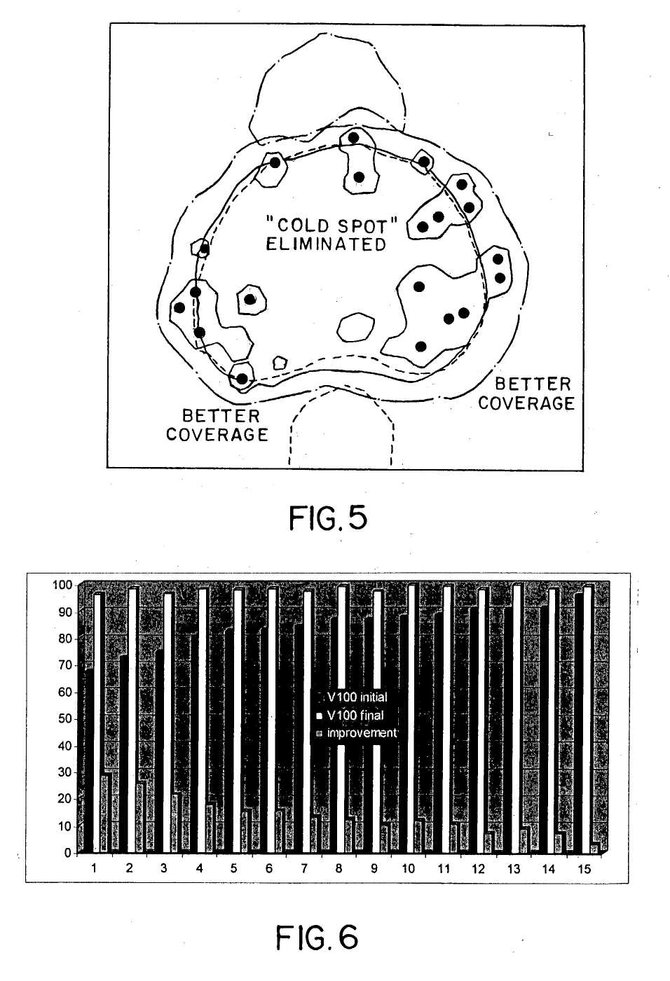

An analysis of the dosimetry improvement by CT-ultrasound directed supplemental seeding was performed. For each case, CT-based dosimetry analyses performed during and after the procedure were directly compared to each other to assess the magnitude and quality of the dosimetry improvement created by the supplemental seeding procedure. A simple reproducible method of CT-based dosimetry analysis is that the final CT-prostate volume has to be equal to or larger than the pre-planned PTV. This method was followed for the final CT-based dosimetry assessment. Comparative dosimetry parameters analyzed included the respective prostate volumes, V100, V150 and D90 values, as well as the respective urethal D90, D50 and D10 values. A graph comparing V100 values from 15 patients is shown in FIG. 6. In each case, substantial improvement was seen after the second round of seed placement. All 15 cases had a final V100 of greater or equal to 90% whereas only four of the cases had a V100 greater or equal to 90% after the first round of seed implantation.

### EXAMPLE

A patient was diagnosed with prostate cancer and determined to be a good candidate for protate brachytherapy. The prognosis in the case was favorable as judged by many parameters well known to those skilled in the art. The patient was scheduled for a number of preoperative tests, including imaging studies, and surgery.

On a preoperative day, ultrasound contours were taken of the prostate. The images were aligned using Variseed® software. A preplan was developed for the implantation of ¹²⁵I seeds in an irregular pattern throughout the prostate to deliver a dose of 145Gy.

On the operative day, the patient was prepped by the nursing staff. The patient was put under conscious sedation and generous local anesthesia to eliminate pain while allowing the patient to be aware of his surroundings. An ultrasound probe was inserted into the rectum and images were used in conjunction with a template to guide the placement of the seeds thoughout the prostate. 150 seeds were implanted, accounting for 90% of the total radioactivity to be used. Upon completion of seed placement, a stepping ultrasound was performed using the inserted ultrasound. The images were transferred to the planning computer for immediate ultrasound-based prostate contouring. The patient was transported to the CT-suite where images were obtained and sent to the same computer that contained the new ultrasound images. After the images were obtained, the patient was returned to the surgical suite and re-prepped for surgery by the nursing staff.

As the patient was being re-prepped for supplemental seeding, the contours for each optical section obtained by CT (dashed line) and ultrasound (solid line) were analyzed to determine the location of the seeds (filled-in circles) and relative to prostate contours as shown schematically in the representative optical section in FIG. 1. In the method of the invention, all of the optical sections are assembled into a three dimensional image for the analysis of seed placement and dosimetry. For the sake of clarity of the drawings, single optical sections are represented in the figures. For further clarity of the drawing, the grids are shown only as numbers and letters along the periphery of the grid. No holes through which needles can be inserted are indicated at the intersection of lines that would be drawn from each of the numbers and letters. Grids are well known to those skilled in the art and the representation in the drawing would be sufficient for understanding the method of the invention.

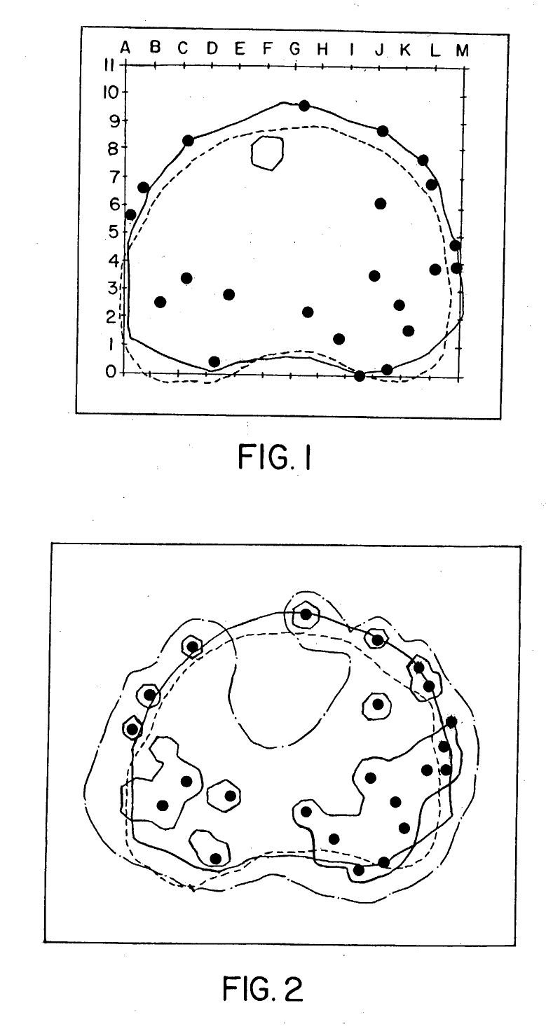

After identification of the seeds, dose lines (alternating dot and dashed line) were calculated and displayed relative to the prostate contours as shown schematically as an optical section in FIG. 2. This calculation was performed using the Variseed® software program. The dose volume analysis of the assembled optical sections revealed that 84% of the prostate was receiving the desired dose of radioactivity (V100=84%). For optimal therapeutic outcomes, it is desirable that at least 90% of the prostate receives the prescribed dose.

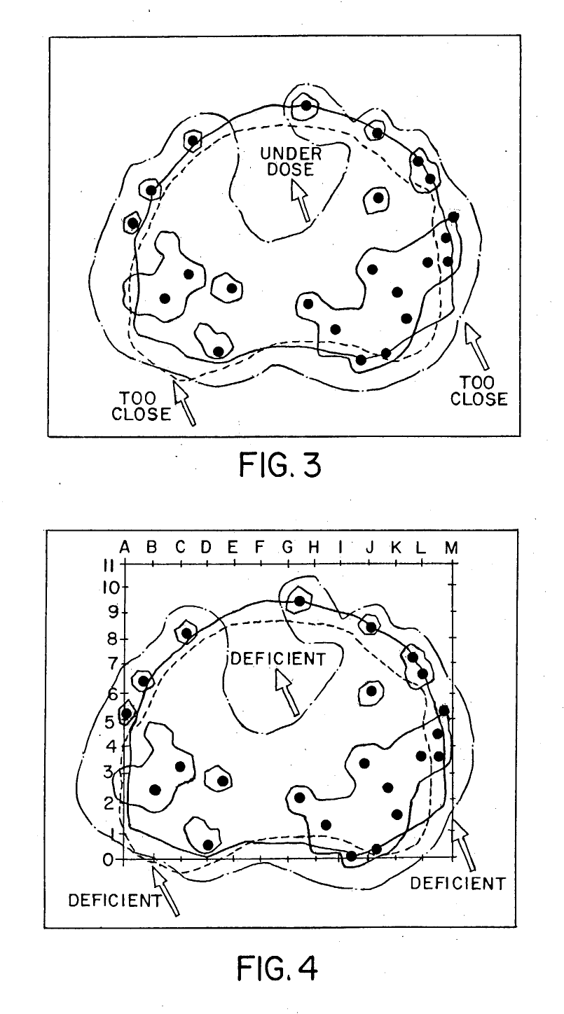

Specific areas of inadequate dosage were identified as shown schematically in the optical section in FIG. 3. An area towards the center of the prostate had received an insufficient dose of radioactivity as indicated. In two regions in the periphery of the prostate, the dose line came too close to the prostate contour lines as indicated.

To definitively locate the regions of receiving an insufficient dose of radioactivity, the image was overlaid on the ultrasound grid as shown in FIG. 4, and the areas of deficient dosage identified were assigned a specific series of x, y and z coordinates. The x and y coordinates were obtained from the grid and the z coordinate was obtained from the location of the optical section. A miniplan was developed for the implantation of seeds at the “cold spots” to correct any deficiencies in dosing.

Simulated seeds were inserted into the images on the computer and a dosage calculation was performed to insure that the insertion of the additional seeds would result in a V100 value greater than 90%. Analysis of the dose distribution with the simulated seeds added increased the V100 value from 84% to 98%. The seeds were implanted as indicated by the miniplan. At the conclusion of the procedure, the patient underwent final ultrasound and CT scans to determine the quality of the overall procedure as shown in the optical section of FIG. 5 which was obtained close to the section used in the previous figures. As the patient cannot be placed in exactly the same location in the scanner, it is essentially impossible to obtain optical sections at identical planes between imaging sessions, though agreement within 2-3 mm is typically possible. The final V100 measurement was found to be 98%.

Although an exemplary embodiment of the invention has been described above by way of example only, it will be understood by those skilled in the field that modifications may be made to the disclosed embodiment without departing from the scope of the invention, which is defined by the appended claims.

We claim:

1. A method for brachytherapy for treatment of cancer in a human or animal having a body comprising:
development of a preplan for implantation of seeds into a region of the body;
implantation of the seeds into the region of the body according to the preplan;
collection of a first image of the region of the body using three-dimensional ultrasonography to detect organs;
collection of a second image of the region of the body using computed tomography to detect implanted seeds and organs;
analysis of the first image and the second image together to determine dose distribution;
development of a miniplan for seed implantation to optimize dose distribution in the region of the body; and
implantation of additional seeds according to the miniplan.

2. The method of claim 1, wherein the method occurs within a single day.

3. The method of claim 1, wherein the method occurs in real time.

4. The method of claim 1, further comprising administration of conscious sedation and local anesthesia.

5. The method of claim 1, further comprising administration of spinal anesthesia.

6. The method of claim 1, wherein the region of the body is prostate.

7. The method of claim 1, wherein the analysis is performed by merging the first image with the second image.

8. The method of claim 7, wherein the seeds in the miniplan are simulated in the merged image.

9. The method as in claim 8, wherein the merged image containing the simulated seeds is used to calculate a dose distribution based on the seeds implanted using the preplan and the miniplan.

10. The method of claim 1, wherein the analysis is performed to identify deficiencies in dosing.

11. The method as in claim 1, wherein the miniplan is developed to correct for deficiencies in dosing.

12. The method as in claim 1, wherein the miniplan is developed to increase the number of seeds near a dominant lesion.

13. The method as in claim 1, wherein the miniplan is developed to place seeds outside of the prostate.

14. The method as in claim 1, wherein the seeds are radioactive.

15. The method as in claim 1, wherein the seeds implanted based on the miniplan comprise less than 50% of the total radioactivity.

16. The method as in claim 1, wherein the seeds implanted based on the miniplan comprise less than 20% of the total radioactivity delivered.

17. The method as in claim 1, wherein the seeds implanted based on the miniplan comprise less than 10% of the total radioactivity.

18. The method as in claim 1, wherein the seed placement is imaged after completion of the miniplan.

19. The method as in claim 1, wherein the in human or animal is monitored for amelioration of cancer.

20. The method according to claim 1, wherein the seed placement is ultrasound guided.
